SEQUENCE LISTING (13 sequences)

SEQ ID NO: 1 - protein, 419 aa, Clostridium tetani
MKGKPITINNFRYSDPVNNDTIIMMEPPYCKGLDIYYKAFKITDRIWIVPERYEFGTKPEDFNPPSSLIEGASEYYDPNYLRTDSDKDRFLQTMVKLFNRIKNNVAGEALLDKIINAIPYLGNSYSLLDKFDTNSNSVSFNLLEQDPSGATTKSAMLTNLIIFGPGPVLNKNEVRGIVLRVDNKNYFPCRDGFGSIMQMAFCPEYVPTFDNVIENITSLTIGKSKYFQDPALLLMHALIHVLHGLYGMQVSSHEGIIPSKQEIYMQHTYPISAEALFTFGGQDANLISIDIKNDLYEKTLNDYKAIANKLSQVTSCNDPNIDIDSYKQIYQQKYQFDKDSNGQYIVNEDKFQILYNSIMYGFTEIELGKKFNIKTRLSYFSMNHDPVKIPNLLDDTIYNDTEGFNIESKDLKSEYKGQN

SEQ ID NO: 2 - protein, 960 aa, Clostridium tetani
MKGKSRVNTNAFRNVDGSGLVSKLIGLCKKIIPPTNIREGNLYNRTASLTDLGGELCIKIKNEDLTFIAEKNSFSEEPFQDEIVSYNTKNKPLNFNYSLDKIILDYNLQSKITLPNDRTTPVTKGIPYAPEYKSNAASTIEIHNIDDNTIYQYLYAQKSPTTLQRITMTNSVDDALINSTKIYSYFPSVISKVNQGAQGGILFLQWVRDIIDDFTNESSQKTTIDKISDVSTIVPYIGPALNIVKQGYEGNFIGALETTGVVLLLEYIPEITLPVIAALSIAESSTGPSKRSFIEDLLFNKVTLAGGTESNKKFLPFQQFGRDIAGQKEKIIKTIDNFLEKRYEKWIEVYKLVKAKWLGTVNTQFQKRSYQMYRSLEYQVDAIKKIIDYEYKIYSGPDGGKEQIADEINNLKNKLEEKANKAMININIFMRSSRSFLVNQMINEAKKQLLEFDTQSKNILMQYIKANSKFIGITELKKLESKINKVFSTPIPFSYSKNLDCWVDNEEDIDVILKKSTILNLDINNDIISDISGFNSSVITYPDAQLVPGINGKAIHLVNNESSEVIVHKAMDIEYNDMFNNFTVSFWLRVPKVSSHLEQYGTNEYSIISSMKKYSLSIGSGWSVSLKGNNLIWTLKDSAGEVRQITFRDLSDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPSYTNGKLNIYYRRLYNGLKFIIKYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTGGDEGWTNDLVPRGSGHHHHHH

SEQ ID NO: 3 - protein, 1306 aa, Clostridium tetani
MKGKPITINNFRYSDPVNNDTIIMMEPPYCKGLDIYYKAFKITDRIWIVPERYEFGTKPEDFNPPSSLIEGASEYYDPNYLRTDSDKDRFLQTMVKLFNRIKNNVAGEALLDKIINAIPYLGNSYSLLDKFDTNSNSVSFNLLEQDPSGATTKSAMLTNLIIFGPGPVLNKNEVRGIVLRVDNKNYFPCRDGFGSIMQMAFCPEYVPTFDNVIENITSLTIGKSKYFQDPALLLMHALIHVLHGLYGMQVSSHEGIIPSKQEIYMQHTYPISAEALFTFGGQDANLISIDIKNDLYEKTLNDYKAIANKLSQVTSCNDPNIDIDSYKQIYQQKYQFDKDSNGQYIVNEDKFQILYNSIMYGFTEIELGKKFNIKTRLSYFSMNHDPVKIPNLLDDTIYNDTEGFNIESKDLKSEYKGQNMRVNTNAFRNVDGSGLVNLYNRTASLTDLGGELCIKIKNEDLTFIAEKNSFSEEPFQDEIVSYNTKNKPLNFNYSLDKIILDYNLQSKITLPNDRTTPVTKGIPYAPEYKSNAASTIEIHNIDDNTIYQYLYAQKSPTTLQRITMTNSVDDALINSTKIYSYFPSVISKVNQGAQGGILFLQWVRDIIDDFTNESSQKTTIDKISDVSTIVPYIGPALNIVKQGYEGNFIGALETTGVVLLLEYIPEITLPVIAALSIAESSTGQKEKIIKTIDNFLEKRYEKWIEVYKLVKAKWLGTVNTQFQKRSYQMYRSLEYQVDAIKKIIDYEYKIYSGPDGKEQIADEINNLKNKLEEKANKAMININIFMRESSRSFLVNQMINEAKKQLLEFDTQSKNILMQYIKANSKFIGITELKKLESKINKVFSTPIPFSYSKNLDCWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYGTNEYSIISSMKKYSLSIGSGWSVSLKGNNLIWTLKDSAGEVRQITFRDLSDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTNDGG

SEQ ID NO: 4 - DNA, 5073 bp, Clostridium tetani
  source 1..5073: mol_type = other DNA
atgaaggggaagcccattaccatcaacaactttcgctactccgaccccgtgaacaacgacaccattattatgatggagcccccgtactgcaaaggcctggacatctactacaaagcgtttaagatcaccgaccggatctggattgtgccggaacgctacgagttcggcactaagccggaggacttcaaccccccatcctccctcattgagggcgcctcggagtactacgacccgaactatctgaggaccgactcagacaaagacagattcctgcagactatggtcaagctgttcaaccggatcaagaacaacgtggctggcgaagccctgctggacaagatcatcaacgcaattccctatctgggcaacgcagcgtacgactcctatatcgtcatcggagtggggaactccgccctcactctgagctactcgctcttggacaagttcgataccaactccaactccgtgtccttcaaccttctggagcaagatccgtcaggagccaccactaagagcgccatgctgactaacctcatcatcttcggccctggtcctgtcctcaacaagaacgaagtccggggaatcgtgctgcgcgtggacaacaagaactacttcccctgtcgggacggtttcggatcgatcatgcaaatggcgttctgccctgaatacgtgccgaccttcgacaacgtcatcgaaaacatcacctctctgaccatcgggaagtcgaagtacttccaggatccagccttgctgctgatgcacgccctgatccatgtgcttcacgggctgtacgggatgcaagtgtcatcccacgaagcggcttacatcgtcattggcgtgggggatagcgctctgggaccaggtcccggagagggaaccgacgccccttgcaagatcccgttctcgagccaggacgagaagggcatcatcccgtccaagcaggaaatctacatgcagcacacctacccgatatctgccgaagccctgtttactttcggaggacaggatgccaatctgatcagtatcgacattaagaacgatctctacgaaaagactcttaacgactacaaggccatcgccaacaagctgtcacaggtcacctcgtgcggacctggacctggacgccatgtgctgggtcggctcattaccgtgaaccccgtgaccgagaagaacgaccctaacatcgacatagatagctacaagcagatctaccagcagaagtaccaattcgataaggactccaacggccagtacattgtgaacgaggataagttccagattctgtacaacagcattatgtacggctttaccgaaatcgagctggggaagaaattcaacattaagacccgcttgagctatttctccatgaaccacgatccagcggcctacagagggatgtcctacgctatgtgtaccaatacgttcgtggtgttaaagaaggaagtgtccgtgaagattcccaacctcctggacgacacaatctacaatgacaccgaaggattcaacattgaatccaaggatctgaagtccgagtacaagggccagaatatgcgggtcaacaccaatgctttccgcaacgtggacggctccggtttggtgtccaagctgatcggcctgtgcaaaaagattattcctccgactaacatcagggagggaaacctgtataacaggaccgccagcctcaccgacctgggcggagaattgtgcatcaagatcaagaacgccgcctacatcgtgattggagtgggggactcagccctgggacctggaccgggagagggcactgatgcgccctgcaaaattcctttctcatcccaagatgaaaaggaagatctgacattcatcgctgagaagaactcgttcagcgaggagccgtttcaggacgaaatcgtgtcctacaacacgaagaataagccgctcaacttcaactactcccttgataagatcatcctggactacaatctccaatccaagattaccctcccgaacgatagaaccacacccgtgaccaagggcattccatacgcgccggagtacaagagcaacgccgcctccaccatcgaaatccacaacattgacgacaataccatctaccagtacctgtacgcacagaagtcccctacgaccctgcagcggattaccatgactaacagcgtggacgacgcgctcatcaacagcactaagatctattcctatttcccctccgtgatttccaaggtcaaccagggagcgcaaggtcccggacctggcagacacgtgcttgggcggctgattaccgtcaatcctgtgaccgaaaagggggggattctcttcctccaatgggtccgcgacatcatcgacgatttcactaacgaatcgtcccaaaagaccactatcgacaagatctccgatgtgtcgaccgctgcctacattgtcatcggcgtcggagactcggcattgggaccaggacccggagaaggcaccgacgcaccgtgcaagattccgttttcgagccaagacgagaagatcgtgccttacattgggccggccctgaacatcgtgaaacagggttacgaggggaacttcattggtgccctggagactacaggagtggtgctgctgctggagtacattccggagatcactctccccgtgattgcggccctgagcatcgccgaaagcagcactgcagcatacagaggaatgtcatatgccatgtgcaccaacaccttcgtggtgctgaaaaaggaagtcagcggaccgtcaaagcggtcgttcatcgaggatctcctgttcaacaaggtcacgctggccggaggaaccgaatccaacaagaagtttctgccgttccaacaattcggacgggacattgcgggccagaaggagaaaatcatcaagaccattgacaacttcctggagaagcgctacgaaaagtggatcgaagtctacaagctcgtcaaagccaagtggcttggcaccgtgaacacccagttccagaagcggtcgtaccagatgtaccggagcctggaataccaggtcgatgcgattaagaagatcatcgactacgagtacaagatctactcgggtcctgatggccctggaccaggcagacatgtactgggtagattgattacagtgaaccctgtgactgagaagggtggaaaggaacagattgctgacgaaatcaacaacctcaagaacaagctggaggaaaaagccaacaaggccatgatcaacatcaacattttcatgagatcctcgcggagcttccttgtgaaccagatgattaacgaagcgaagaagcagctcttggaattcgacacccagtccaagaacatactgatgcagtacatcaaggccaactccaagttcatcgggatcaccgaactcaagaagctggagtccaagatcaacaaagtgttctcgacccccattcccttctcgtactccaagaacctggactgttgggtcgataacgaggaggatatcgacgtgatcctcaagaagtccaccattttgaacctggatatcaacaacgacatcatcagcgacatcagcggattcaactcatcggtgatcacctaccccgacgcccagttggtccctgggatcaatggaaaggccatccacctcgtgaacaatgagtcctccgaagtgatcgtgcacaaagccatggccgcatatcggggcatgagctacgccatgtgtactaacacttttgtcgtgctgaagaaagaagtgtccgatattgagtacaatgatatgttcaacaacttcaccgtgtcattttggctccgcgtgccgaaagtgtcctcacaccttgccgcctatcggggaatgtcgtacgcgatgtgcacgaacactttcgtggtcctcaaaaaggaggtgtccgagcagtacggcaccaacgaatactccatcatctcatccatgaagaagtactcgctgtccattggtagcgggtggtcggtgtcactgaagggcaacaaccttatctggactctgaaagactcggccggagaagtccggcagatcacctttagggacctgtccgacaagttcaacgcctacttggcgaataagtgggtgttcatcactatcacgaatgaccgcctgagctccgcaaatctgtacattaacggcgtgcttatgggctccgccgaaattaccggtctgggggccatcagagaggacaacaacattaccctgaagctggaccgctgcaacaacaacaatcagtacgttagcatcgacaagttccgaattttctgcaaggccctgaatcccaaggagatcgaaaagctgtacactagttacctctccatcaccttccttcgggacttctgggggaatccccttcgctacgataccgaatattacctcatcccggtggcatcgagctctaaggacgtgcaactgaagaacatcactgactatatgtacctgaccaacgccccctcctacactaacggaaagctgaacatctattaccggaggctgtacaacggactcaagttcatcatcaagtacacccccaacaacgaaatcgattccttcgtcaagtccggagatttcatcaagctctacgtgtcgtacaacaacaacgagcacatcgtgggatacccgaaggacggaaacgctttcaacaacctggatcgcattctgcgcgtcggatataacgctccgggcatcccactatacaagaagatggaggccgtgaagctgcgggatttgaaaacttacagcgtgcagctgaagctgtacgacgacaagaatgcctccctgggactcgtgggcactcacaacggccagatcggaaacgaccccaatcgggacatcctcatcgcctccaactggtacttcaaccacctgaaggataagattctgggttgcgattggtactttgtcccggccgcttacattgtgattggtgtcggagacagcgcactgggtccaggacctggcgaagggactgacgccccgtgcaaaatcccgtttagctcgcaagacgaaaaagatgagggttggactaacgacctcgtgccaagaggaagcggccatcaccaccaccatcatcaccatcaccactgataa

SEQ ID NO: 5 - protein, 1689 aa, Clostridium tetani
MKGKPITINNFRYSDPVNNDTIIMMEPPYCKGLDIYYKAFKITDRIWIVPERYEFGTKPEDFNPPSSLIEGASEYYDPNYLRTDSDKDRFLQTMVKLFNRIKNNVAGEALLDKIINAIPYLGNAAYDSYIVIGVGNSALTLSYSLLDKFDTNSNSVSFNLLEQDPSGATTKSAMLTNLIIFGPGPVLNKNEVRGIVLRVDNKNYFPCRDGFGSIMQMAFCPEYVPTFDNVIENITSLTIGKSKYFQDPALLLMHALIHVLHGLYGMQVSSHEAAYIVIGVGDSALGPGPGEGTDAPCKIPFSSQDEKGIIPSKQEIYMQHTYPISAEALFTFGGQDANLISIDIKNDLYEKTLNDYKAIANKLSQVTSCGPGPGRHVLGRLITVNPVTEKNDPNIDIDSYKQIYQQKYQFDKDSNGQYIVNEDKFQILYNSIMYGFTEIELGKKFNIKTRLSYFSMNHDPAAYRGMSYAMCTNTFVVLKKEVSVKIPNLLDDTIYNDTEGFNIESKDLKSEYKGQNMRVNTNAFRNVDGSGLVSKLIGLCKKIIPPTNIREGNLYNRTASLTDLGGELCIKIKNAAYIVIGVGDSALGPGPGEGTDAPCKIPFSSQDEKEDLTFIAEKNSFSEEPFQDEIVSYNTKNKPLNFNYSLDKIILDYNLQSKITLPNDRTTPVTKGIPYAPEYKSNAASTIEIHNIDDNTIYQYLYAQKSPTTLQRITMTNSVDDALINSTKIYSYFPSVISKVNQGAQGPGPGRHVLGRLITVNPVTEKGGILFLQWVRDIIDDFTNESSQKTTIDKISDVSTAAYIVIGVGDSALGPGPGEGTDAPCKIPFSSQDEKIVPYIGPALNIVKQGYEGNFIGALETTGVVLLLEYIPEITLPVIAALSIAESSTAAYRGMSYAMCTNTFVVLKKEVSGPSKRSFIEDLLFNKVTLAGGTESNKKFLPFQQFGRDIAGQKEKIIKTIDNFLEKRYEKWIEVYKLVKAKWLGTVNTQFQKRSYQMYRSLEYQVDAIKKIIDYEYKIYSGPDGPGPGRHVLGRLITVNPVTEKGGKEQIADEINNLKNKLEEKANKAMININIFMRSSRSFLVNQMINEAKKQLLEFDTQSKNILMQYIKANSKFIGITELKKLESKINKVFSTPIPFSYSKNLDCWVDNEEDIDVILKKSTILNLDINNDIISDISGFNSSVITYPDAQLVPGINGKAIHLVNNESSEVIVHKAMAAYRGMSYAMCTNTFVVLKKEVSDIEYNDMFNNFTVSFWLRVPKVSSHLAAYRGMSYAMCTNTFVVLKKEVSEQYGTNEYSIISSMKKYSLSIGSGWSVSLKGNNLIWTLKDSAGEVRQITFRDLSDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPSYTNGKLNIYYRRLYNGLKFIIKYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPAAYIVIGVGDSALGPGPGEGTDAPCKIPFSSQDEKDEGWTNDLVPRGSGHHHHHHHHHH

SEQ ID NO: 6 - DNA, 1572 bp, Clostridium tetani
  source 1..1572: mol_type = other DNA
atgaaggggaagcccattaccatcaacaactttcgctactccgaccccgtgaacaacgacaccattattatgatggagcccccgtactgcaaaggcctggacatctactacaaagcgtttaagatcaccgaccggatctggattgtgccggaacgctacgagttcggcactaagccggaggacttcaaccccccatcctccctcattgagggcgcctcggagtactacgacccgaactatctgaggaccgactcagacaaagacagattcctgcagactatggtcaagctgttcaaccggatcaagaacaacgtggctggcgaagccctgctggacaagatcatcaacgcaattccctatctgggcaacgcagcgtacgactcctatatcgtcatcggagtggggaactccgccctcactctgagctactcgctcttggacaagttcgataccaactccaactccgtgtccttcaaccttctggagcaagatccgtcaggagccaccactaagagcgccatgctgactaacctcatcatcttcggccctggtcctgtcctcaacaagaacgaagtccggggaatcgtgctgcgcgtggacaacaagaactacttcccctgtcgggacggtttcggatcgatcatgcaaatggcgttctgccctgaatacgtgccgaccttcgacaacgtcatcgaaaacatcacctctctgaccatcgggaagtcgaagtacttccaggatccagccttgctgctgatgcacgccctgatccatgtgcttcacgggctgtacgggatgcaagtgtcatcccacgaagcggcttacatcgtcattggcgtgggggatagcgctctgggaccaggtcccggagagggaaccgacgccccttgcaagatcccgttctcgagccaggacgagaagggcatcatcccgtccaagcaggaaatctacatgcagcacacctacccgatatctgccgaagccctgtttactttcggaggacaggatgccaatctgatcagtatcgacattaagaacgatctctacgaaaagactcttaacgactacaaggccatcgccaacaagctgtcacaggtcacctcgtgcggacctggacctggacgccatgtgctgggtcggctcattaccgtgaaccccgtgaccgagaagaacgaccctaacatcgacatagatagctacaagcagatctaccagcagaagtaccaattcgataaggactccaacggccagtacattgtgaacgaggataagttccagattctgtacaacagcattatgtacggctttaccgaaatcgagctggggaagaaattcaacattaagacccgcttgagctatttctccatgaaccacgatccagcggcctacagagggatgtcctacgctatgtgtaccaatacgttcgtggtgttaaagaaggaagtgtccgtgaagattcccaacctcctggacgacacaatctacaatgacaccgaaggattcaacattgaatccaaggatctgaagtccgagtacaagggccagaatcatcaccaccaccaccactgataa

SEQ ID NO: 7 - protein, 497 aa, Clostridium tetani
MKGKPITINNFRYSDPVNNDTIIMMEPPYCKGLDIYYKAFKITDRIWIVPERYEFGTKPEDFNPPSSLIEGASEYYDPNYLRTDSDKDRFLQTMVKLFNRIKNNVAGEALLDKIINAIPYLGNAAYDSYIVIGVGNSALTLSYSLLDKFDTNSNSVSFNLLEQDPSGATTKSAMLTNLIIFGPGPVLNKNEVRGIVLRVDNKNYFPCRDGFGSIMQMAFCPEYVPTFDNVIENITSLTIGKSKYFQDPALLLMHALIHVLHGLYGMQVSSHEAAYIVIGVGDSALGPGPGEGTDAPCKIPFSSQDEKGIIPSKQEIYMQHTYPISAEALFTFGGQDANLISIDIKNDLYEKTLNDYKAIANKLSQVTSCGPGPGRHVLGRLITVNPVTEKNDPNIDIDSYKQIYQQKYQFDKDSNGQYIVNEDKFQILYNSIMYGFTEIELGKKFNIKTRLSYFSMNHDPAAYRGMSYAMCTNTFVVLKKEVSVKIPNLLDDTIYND

SEQ ID NO: 8 - DNA, 3476 bp, Clostridium tetani
  source 1..3476: mol_type = other DNA
atgaagggcaagtcccgggtcaacaccaatgctttccgcaacgtggacggctccggtttggtgtccaagctgatcggcctgtgcaaaaagattattcctccgactaacatcagggagggaaacctgtataacaggaccgccagcctcaccgacctgggcggagaattgtgcatcaagatcaagaacgccgcctacatcgtgattggagtgggggactcagccctgggacctggaccgggagagggcactgatgcgccctgcaaaattcctttctcatcccaagatgaaaaggaagatctgacattcatcgctgagaagaactcgttcagcgaggagccgtttcaggacgaaatcgtgtcctacaacacgaagaataagccgctcaacttcaactactcccttgataagatcatcctggactacaatctccaatccaagattaccctcccgaacgatagaaccacacccgtgaccaagggcattccatacgcgccggagtacaagagcaacgccgcctccaccatcgaaatccacaacattgacgacaataccatctaccagtacctgtacgcacagaagtcccctacgaccctgcagcggattaccatgactaacagcgtggacgacgcgctcatcaacagcactaagatctattcctatttcccctccgtgatttccaaggtcaaccagggagcgcaaggtcccggacctggcagacacgtgcttgggcggctgattaccgtcaatcctgtgaccgaaaagggggggattctcttcctccaatgggtccgcgacatcatcgacgatttcactaacgaatcgtcccaaaagaccactatcgacaagatctccgatgtgtcgaccgctgcctacattgtcatcggcgtcggagactcggcattgggaccaggacccggagaaggcaccgacgcaccgtgcaagattccgttttcgagccaagacgagaagatcgtgccttacattgggccggccctgaacatcgtgaaacagggttacgaggggaacttcattggtgccctggagactacaggagtggtgctgctgctggagtacattccggagatcactctccccgtgattgcggccctgagcatcgccgaaagcagcactgcagcatacagaggaatgtcatatgccatgtgcaccaacaccttcgtggtgctgaaaaaggaagtcagcggaccgtcaaagcggtcgttcatcgaggatctcctgttcaacaaggtcacgctggccggaggaaccgaatccaacaagaagtttctgccgttccaacaattcggacgggacattgcgggccagaaggagaaaatcatcaagaccattgacaacttcctggagaagcgctacgaaaagtggatcgaagtctacaagctcgtcaaagccaagtggcttggcaccgtgaacacccagttccagaagcggtcgtaccagatgtaccggagcctggaataccaggtcgatgcgattaagaagatcatcgactacgagtacaagatctactcgggtcctgatggccctggaccaggcagacatgtactgggtagattgattacagtgaaccctgtgactgagaagggtggaaaggaacagattgctgacgaaatcaacaacctcaagaacaagctggaggaaaaagccaacaaggccatgatcaacatcaacattttcatgagatcctcgcggagcttccttgtgaaccagatgattaacgaagcgaagaagcagctcttggaattcgacacccagtccaagaacatactgatgcagtacatcaaggccaactccaagttcatcgggatcaccgaactcaagaagctggagtccaagatcaacaaagtgttctcgacccccattcccttctcgtactccaagaacctggactgttgggtcgataacgaggaggatatcgacgtgatcctcaagaagtccaccattttgaacctggatatcaacaacgacatcatcagcgacatcagcggattcaactcatcggtgatcacctaccccgacgcccagttggtccctgggatcaatggaaaggccatccacctcgtgaacaatgagtcctccgaagtgatcgtgcacaaagccatggccgcatatcggggcatgagctacgccatgtgtactaacacttttgtcgtgctgaagaaagaagtgtccgatattgagtacaatgatatgttcaacaacttcaccgtgtcattttggctccgcgtgccgaaagtgtcctcacaccttgccgcctatcggggaatgtcgtacgcgatgtgcacgaacactttcgtggtcctcaaaaaggaggtgtccgagcagtacggcaccaacgaatactccatcatctcatccatgaagaagtactcgctgtccattggtagcgggtggtcggtgtcactgaagggcaacaaccttatctggactctgaaagactcggccggagaagtccggcagatcacctttagggacctgtccgacaagttcaacgcctacttggcgaataagtgggtgttcatcactatcacgaatgaccgcctgagctccgcaaatctgtacattaacggcgtgcttatgggctccgccgaaattaccggtctgggggccatcagagaggacaacaacattaccctgaagctggaccgctgcaacaacaacaatcagtacgttagcatcgacaagttccgaattttctgcaaggccctgaatcccaaggagatcgaaaagctgtacactagttacctctccatcaccttccttcgggacttctgggggaatccccttcgctacgataccgaatattacctcatcccggtggcatcgagctctaaggacgtgcaactgaagaacatcactgactatatgtacctgaccaacgccccctcctacactaacggaaagctgaacatctattaccggaggctgtacaacggactcaagttcatcatcaagtacacccccaacaacgaaatcgattccttcgtcaagtccggagatttcatcaagctctacgtgtcgtacaacaacaacgagcacatcgtgggatacccgaaggacggaaacgctttcaacaacctggatcgcattctgcgcgtcggatataacgctccgggcatcccactatacaagaagatggaggccgtgaagctgcgggatttgaaaacttacagcgtgcagctgaagctgtacgacgacaagaatgcctccctgggactcgtgggcactcacaacggccagatcggaaacgaccccaatcgggacatcctcatcgcctccaactggtacttcaaccacctgaaggataagattctgggttgcgattggtactttgtcccggccgcttacattgtgattggtgtcggagacagcgcactgggtccaggacctggcgaagggactgacgccccgtgcaaaatcccgtttagctcgcaagacgaaaaagatgagggttggactaa

SEQ ID NO: 9 - protein, 1173 aa, Clostridium tetani
MKGKSRVNTNAFRNVDGSGLVSKLIGLCKKIIPPTNIREGNLYNRTASLTDLGGELCIKIKNAAYIVIGVGDSALGPGPGEGTDAPCKIPFSSQDEKEDLTFIAEKNSFSEEPFQDEIVSYNTKNKPLNFNYSLDKIILDYNLQSKITLPNDRTTPVTKGIPYAPEYKSNAASTIEIHNIDDNTIYQYLYAQKSPTTLQRITMTNSVDDALINSTKIYSYFPSVISKVNQGAQGPGPGRHVLGRLITVNPVTEKGGILFLQWVRDIIDDFTNESSQKTTIDKISDVSTAAYIVIGVGDSALGPGPGEGTDAPCKIPFSSQDEKIVPYIGPALNIVKQGYEGNFIGALETTGVVLLLEYIPEITLPVIAALSIAESSTAAYRGMSYAMCTNTFVVLKKEVSGPSKRSFIEDLLFNKVTLAGGTESNKKFLPFQQFGRDIAGQKEKIIKTIDNFLEKRYEKWIEVYKLVKAKWLGTVNTQFQKRSYQMYRSLEYQVDAIKKIIDYEYKIYSGPDGPGPGRHVLGRLITVNPVTEKGGKEQIADEINNLKNKLEEKANKAMININIFMRSSRSFLVNQMINEAKKQLLEFDTQSKNILMQYIKANSKFIGITELKKLESKINKVFSTPIPFSYSKNLDCWVDNEEDIDVILKKSTILNLDINNDIISDISGFNSSVITYPDAQLVPGINGKAIHLVNNESSEVIVHKAMAAYRGMSYAMCTNTFVVLKKEVSDIEYNDMFNNFTVSFWLRVPKVSSHLAAYRGMSYAMCTNTFVVLKKEVSEQYGTNEYSIISSMKKYSLSIGSGWSVSLKGNNLIWTLKDSAGEVRQITFRDLSDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPSYTNGKLNIYYRRLYNGLKFIIKYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPAAYIVIGVGDSALGPGPGEGTDAPCKIPFSSQDEKDEGWTNDLVPRGSGHHHHHH

SEQ ID NO: 10 - protein, 35 aa, Dengue virus
AAYIVIGVGDSALGPGPGEGTDAPCKIPFSSQDEK

SEQ ID NO: 11 - protein, 21 aa, Dengue virus
GPGPGRHVLGRLITVNPVTEK

SEQ ID NO: 12 - protein, 23 aa, Dengue virus
AAYRGMSYAMCTNTFVVLKKEVS

SEQ ID NO: 13 - protein, 18 aa, Dengue virus
AAYDSYIVIGVGNSALTL

(19) United States
(12) Patent Application Publication
(10) Pub. No.: US 2024/0424080 A1
(43) Pub. Date: Dec. 26, 2024

(54) Multi-Epitope Vaccine Platform

(71) Applicant: Institute of Advanced Sciences, North Dartmouth, MA (US)

(72) Inventors: Bal Ram Singh, Rehoboth, MA (US); Raj Kumar, Dartmouth, MA (US)

(73) Assignee: Institute of Advanced Sciences, North Dartmouth, MA (US)

(21) Appl. No.: 18/338,054

(22) Filed: Jun. 20, 2023

(51) Int. Cl. A61K 39/12; C12N 15/86

(52) U.S. Cl. CPC A61K 39/12; C12N 15/86; A61K 2039/53; A61K 2039/55505; A61K 2039/55544; A61K 2039/575; A61K 2039/70; C12N 2770/24122; C12N 2770/24134; C12N 2770/24143; C12N 2770/24151; C12N 2770/24171

(57) ABSTRACT

The present invention comprises of a vaccine technology to produce one or more novel vaccine compositions, method of making, and administering, for protecting against at least two of the four serotypes of the dengue virus. The vaccine technology is based on four important ideas: a) using a backbone for a vaccine candidate comprising detoxified tetanus neurotoxin—DrTeNT; b) selecting epitopes that can activate both B-cells and T-cells in a patient to whom the vaccine is administered to provide long term immunity; c) immunizing against all the four serotypes of dengue; and d) an oral/sublingual/buccal/nasal delivery platform and formulation.

### REFERENCE TO SEQUENCE LISTING SUBMITTED ELECTRONICALLY

The official copy of the sequence listing is submitted electronically via EFS-Web as an XML file in compliance with the ST26 standard named “Dengue Patent sequence listing ST26 17 May 23.xml” created on May 17, 2023 and having a size of 27 kilobytes. The sequence listing is filed concurrently with the specification. The sequence listing is part of the specification and is incorporated by reference herein in its entirety.

### FIELD OF THE INVENTION

The present invention relates to a protein vaccine, using a recombinant protein cloning, expression and purification technology. The epitopes are embedded in the recombinant protein (pre-immunized tetanus vaccine platform) carrier, which would not result in any unwanted immune response.

### BACKGROUND OF THE INVENTION

Dengue is a viral illness caused by the bite of a female mosquito carrying the dengue virus. This viral infection is transmitted mainly by the female mosquito of Aedes aegypti species of mosquito, and to a lesser extent by Ae. Albopictus. The virus is called DENV, and it belongs to the vector-borne single positive-strand RNA virus family Flaviviridae (genus Flavivirus). The genus of Flavivirus includes: the West Nile virus, dengue virus, tick-borne encephalitis virus, yellow fever virus, Japanese encephalitis virus, Usutu virus, Zika virus and several other viruses, infection of which results in encephalitis, vascular shock syndrome, acute flaccid paralysis, congenital abnormalities and fetal death. Infection of these viruses have an endemic potential that is due to their insect vectors, breeding habitats, travel, environmental condition and geographical expansion. Beyond arthropod, flaviviruses are known to infect several animal species. Because of widespread distribution of mosquito vectors coupled with several strains of all serotypes, about 2.5 billion people are at risk of dengue infection. The World Health Organization (WHO) considers dengue as one of the fastest growing arboviral diseases (WHO, 2013).

There are four types of Dengue viruses. All of them are distinct viruses without cross immunity. After the initial infection (primary infection) with one serotype of Dengue virus, the person can have life-long immunity against that serotype. However, it does not provide the protection from the secondary infection from another heterologous serotype (DENV-1, DENV-2, DENV-3, and DENV-4). A person can have up to four dengue infections during his lifetime. 40-80% of all dengue infections are asymptomatic, or relatively mild disease follows after the infection. About 5% of infection leads to what is known as Dengue Hemorrhagic Fever (DHF). Patients with DHF can develop an irreversible Dengue Shock Syndrome (DSS), which is fatal. In addition to the Dengue Fever (DF), DHF and DSS, there are other complicated clinical manifestations that can be associated with the infection, such as encephalitis, myocarditis, hepatitis, cholecystitis, myelitis and acute colitis (Ling et al., 2007; Gulati and Maheswari, 2007; Kumar et al., 2008; Wasay et al., 2008; Solomon et al., 2000; Park et al., 2008). Among the several possibilities that have been identified, secondary infections by heterologous serotypes present a significant risk factor. Additionally, in the absence of heterologous neutralizing antibodies, heterologous cross-reactive antibodies may form complexes with the virus and enhance binding to the cells.

Currently, there are no dengue vaccines or antiviral drugs against these viruses available for the public use. Several candidate vaccines, e.g., live-attenuated, inactivated whole virus and recombinant vaccines are in the process of development. Since all of the four serotypes of the Dengue are sufficiently antigenically different, it is a necessary to have a tetravalent vaccine or four monovalent vaccines. However, the major problem associated with this type of vaccine is that the Dengue virus present in infected person leads to the problem of antibody mediated enhancement (ADE). A few promising approaches to produce vaccine are encouraging, including genetic modification to attenuate the dengue virus and production of live attenuated virus in primary dog kidney cells (Edelmen et al., 2003; Simasathien et al., 2008. Sun et al., 2009), but they are associated with safety concerns, such as potential reactogenicity, interference amongst the viruses, possible reversion to native virus, and possible increase of virus infectivity. Therefore, the development of an efficient vaccine for dengue has some serious challenges such as the following.
A) The existence of four antigenically different, yet related serotypes.
B) A poor understanding of the biology and the pathogenesis, and the pathogenesis of DHF and DSS.
C) The need for long term protection.
D) A poor understanding of protective immunity.
E) A lack of a proper model to study the immune response and the mechanism of protection.
F) A need for vaccine trials in different geographical regions to evaluate efficacy of vaccine.
G) The development of a safe and affordable vaccine.

By considering the disadvantages or the challenges a non-replicating vaccine has, then a protein subunit multi-epitopes vaccine could eliminate the risk of reversion. However, protein subunit vaccines are not very potent immunogens and they require suitable adjuvants, or chimeric multi-epitopes, or multi-valent vaccines that can overcome these potential hurdles.

The Dengue virus particle is composed of a nucleocapsid and an envelope. There are three structural proteins (capsid C, precursor membrane prM, and envelope E) and seven nonstructural (NS1, NS2A, NS2B, NS3, NS4A, NS4B and NS5) proteins. The envelope protein E is large (i.e., 495 amino acid residues). The E protein is responsible for binding to the host cell receptors, fusion and entry to the cells. Additionally, E protein plays an important role in protective immunity and neutralizing antibodies (Henchal and Putnak 1990; Lindenbach 2001). E protein consists of three structurally distinct domains (domains are region of polypeptide that is self-stabilizing and can fold independently of other regions of a protein): a central domain (EDI), a dimerization domain (EDII), and an immunoglobulin (Ig)-like C terminal domain (EDIII) (Modis et al., 2003; Kuhn et al., 2002). EDIII spans from amino acid residues 300-400 of C-terminal of E protein, which is exposed and accessible to the virus surface. Several observations suggested that EDIII-based vaccine can evoke protective neutralizing antibodies (Chen et al., 2007; Fahimi et al., 2014; Leng et al., 2009). Anti-EDIII monoclonal antibodies are shown to block dengue infectivity very effectively (Zhang et al., 2011; Crill and Roehrig 2001; Thullier et al., 2001). Neutralizing epitopes (epitopes are sequence of amino acids that are recognized by the immune system) of EDIII domains are identified in the region of 333-351 and 383-389 (Modis et al., 2005). On the other hand, observations are made about ineffectiveness of anti-EDIII antibodies (Wahala et al., 2009; Li et al., 2013), which can be concluded that the whole EDIII based vaccines are not enough to induce protective immunity.

Thus, the object of the presently claimed invention is to provide an effective protein-based vaccine against dengue infection.

### SUMMARY OF THE INVENTION

It was experimentally found by the Applicant herein that the protein obtained using a recombinant protein cloning, expression and purification technology was sufficient for induction of neutralizing antibodies in a subject (patient) against two or more dengue virus serotypes and can act as vaccine to provide protection against dengue infection.

Accordingly, the first aspect of the presently claimed invention is one or more compositions, and a method of treatment for induction of neutralizing antibodies in a subject against two or more dengue virus serotypes, the one or more compositions comprising at least one protein obtained from one or more of the following: a vector embedded with a nucleic acid sequence comprising SEQ ID NO: 4 (nucleotide sequence of Dengue Vaccine Full Length-DeNFL); and/or a vector embedded with a nucleic acid sequence comprising SEQ ID NO: 6; and/or a vector embedded with a nucleic acid sequence comprising SEQ ID NO: 8.

The present invention further comprises a cell transfected with a vector embedded with a nucleic acid sequence comprising SEQ ID NOS: 4, 6, or 8.

As used herein, “DeNFL” refers to the (plasmid) construct of FIG. 1, wherein the abbreviation stands for Dengue Vaccine Full Length (SEQ ID NOS: 4 and 5):

As used herein, “DeNLC” refers to the construct of FIG. 2, wherein the abbreviation stands for Dengue Vaccine Light Chain (SEQ ID NOS: 6 and 7):

As used herein, “DeNHC” refers to the construct of FIG. 3, wherein the abbreviation stands for Dengue Vaccine Heavy Chain (SEQ ID NOS: 8 and 9):

As used herein, “DENV1, DENV2, DENV3” refers to dengue virus serotypes 1, 2, or 3.

The present invention further comprises a protein with at least 85%, 90%, or 95% homology to the amino acid sequences of SEQ ID NOS: 4 and 5 (DeNFL), 6 and 7 (DeNLC), and/or 8 and 9 (DeNHC), where the lower number is the nucleotide sequence, and the higher number is the amino acid sequence.

The second aspect of the presently claimed invention is directed to a method of inducing protection against two or more serotypes of dengue fever in a patient, comprising administering one or more of the compositions of first aspect.

The third aspect of the presently claimed invention is to provide a method of producing a protein according to the first aspect comprising at least one of the following steps of:
a. providing a vector encoding a protein comprising a mutated recombinant light chain of tetanus neurotoxin (˜50 kDa; SEQ. ID NO: 6; DeNLC), or encoding a protein comprising a recombinant heavy chain of tetanus neurotoxin (˜100 kDa; SEQ. ID NO: 9; DeNHC), or encoding a protein comprising a detoxified mutated recombinant full length tetanus neurotoxin (˜150 kDa; SEQ. ID NO: 5; DeNFL);
b. vector constructs of DeNLC, DeNHC and DeNFL contains the one or more of embedded nucleotide sequence of SEQ ID NOS: 10, 11, 12 and 13.
c. obtain the vector constructs of DeNFL (FIG. 1), DeNLC (FIG. 2), and DeNHC (FIG. 3) comprising nucleotide sequences SEQ ID NOS: 4, 6, and/or 8; and
d. obtaining a protein from the vector comprising SEQ ID NOS: 5, 7, and/or 9.

The fourth aspect of the presently claimed invention is a composition for eliciting an effective immune response in a patient against two or more dengue virus serotypes, the composition comprising one or more protein or nucleotide sequences, obtained from one or more of:
a. a vector embedded with a nucleic acid sequence comprising SEQ ID NO: 4; and/or
b. a vector embedded with a nucleic acid sequence comprising SEQ ID NO: 6; and/or,
c. a vector embedded with a nucleic acid sequence comprising SEQ ID NO: 8; and/or,
d. a method for eliciting neutralizing antibody titer against each of the four dengue virus serotypes DENV-1, DENV-2, DENV-3 and DENV-4 in a subject.

The fifth aspect of the presently claimed invention is a composition in which one or more epitopes or domains of dengue virus, or one or more of the nucleic acid sequences encoded by SEQ ID NOS: 4-9, are either embedded in or chemically attached to the sequence encoded by SEQ ID NOS: 1, 2, or 3.

The sixth aspect of the presently claimed invention is a composition in which the vector is embedded with a nucleic acid sequence having at least 85% sequence identity with SEQ ID NO: 4, 6, or 8.

The seventh aspect of the presently claimed invention is the composition in which the nucleic acid sequence is expressed in one or more of: bacteria, yeast, insects, mammalian or any other appropriate expression system.

The eighth aspect of the presently claimed invention is a composition that further comprises one or more proteins derived from Botulinum Neurotoxin (BoNT) produced by a Clostridium botulinum, the one or more proteins comprising: a neurotoxin associated proteins (NAPs); a hemagglutinin (Hn); an open reading frame (Orf); or a combination thereof.

The ninth aspect of the presently claimed invention is a composition that further comprises an adjuvant comprising one or more of: alum, polylactic acid, polyglycolic acid, vitamins, colloidal particles, and non-ionic surfactants.

The tenth aspect of the presently claimed invention is a composition that further comprises one or more of: nanoparticles, emulsions, lipids, liposomes, microspheres, and bioenhancers.

The eleventh aspect of the presently claimed invention is a composition that is able to be administered as a protein, and/or DNA and/or a plasmid molecule.

The twelfth aspect of the presently claimed invention is a composition that is able to be administered as a plasmid molecule alone or in combination with other adjuvants via a route of administration comprising: intraperitoneal (IP), subcutaneous (SubQ), intramuscular (IM), oral, buccal, sub lingual, nasal or topical route.

The thirteenth aspect of the presently claimed invention is a method of inducing protection against two or more serotypes of dengue virus in a patient, comprising administering to a patient a first composition comprising one or more DNA vectors or proteins.

The fourteenth aspect of the presently claimed invention is a first composition comprising one or more proteins encoded by the nucleotide sequence of SEQ ID NOS: 4, 6, or 8, and the one or more proteins are purified to form a stable second composition, which is administered to a patient.

The fifteenth aspect of the presently claimed invention is the method of purifying a protein vaccine that comprises using one or more of: affinity chromatography, ion exchange and gel filtration chromatography.

The sixteenth aspect of the presently claimed invention is a method of treatment, wherein the stable second composition is administered to a patient, and elicits neutralizing B-cell and T-cell responses in the patient against at least two dengue virus serotypes.

The seventeenth aspect of the presently claimed invention is a route of administration of one or more compositions disclosed herein, the route comprising: intraperitoneal (IP), subcutaneous (SubQ), intramuscular (IM), oral, buccal, sub lingual, nasal or topical route.

The eighteenth aspect of the presently claimed invention is a method of producing a protein that is able to elicit an effective immune response against two or more dengue virus serotypes, comprising the steps of:
a. preparing a vector encoding a protein comprising a mutated recombinant light chain of tetanus neurotoxin (˜50 kDa; SEQ. ID NO: 1; DrTeNTLC), or encoding a protein comprising a recombinant heavy chain of tetanus neurotoxin (˜100 kDa; SEQ. ID NO: 2; DrTeNTC), or encoding a protein comprising a detoxified mutated recombinant full length tetanus neurotoxin (˜150 kDa; SEQ. ID NO: 3; DrTeNTFL);
b. embedding the epitopes of constructs DeNFL (FIG. 1), DeNLC (FIG. 2), or DeNHC (FIG. 3) into the vector to obtain a vector comprising nucleotide sequences at least 90% homologous to SEQ ID NOS: 4, 6, and/or 8; and/or
c. obtaining a protein from the vector, the protein comprising an amino acid sequence at least 90% homologous to SEQ ID NOS: 5, 7, and/or 9.

The nineteenth aspect of the presently claimed invention is the method of producing a protein, wherein the vector is the construct of FIG. 1, 2, or 3.

The twentieth aspect of the presently claimed invention is the vector constructs of FIGS. 1, 2, and 3.

The twenty first aspect of the presently claimed invention is the method of producing a protein, wherein the epitope (embedded in the backbone) comprises the amino acid sequence of one or more of: DENV1 (SEQ ID NO: 10); DENV2 (SEQ ID NO: 11); DENV3 (SEQ ID NO: 12); and DENV4 (SEQ ID NO: 13).

The twenty second aspect of the presently claimed invention is the method of producing a protein that is able to elicit an effective immune response against two or more dengue virus serotypes, wherein the protein is expressed in vivo or in vitro.

### BRIEF DESCRIPTION OF THE DRAWINGS

FIG. 1: Mammalian plasmid design of construct I (DeNFL; D2022I) with CMV promoter and several enhancing elements. At the cloning site nucleotide sequence of target protein (DeNFL) was inserted. The target sequence has 10-His amino acid residues at the c-terminus for purification. The target protein size from this construct is ˜190 kDa. The plasmid has two selection markers; kanamycin and puromycin.

FIG. 2: Mammalian plasmid design of construct II (DeNLC; D2022II) with CMV promoter and several enhancing elements. At the cloning site nucleotide sequence of target protein (DeNLC) was inserted. The target sequence has 6-His amino acid at the C-terminus for purification. The target protein size from this construct is ˜58 kDa. The plasmid has two selection markers: kanamycin and puromycin.

FIG. 3: Mammalian plasmid design of construct III (DeNHC; D2022III) with CMV promoter and several enhancing elements. At the cloning site nucleotide sequence of target protein (DeNHC) was inserted. The target sequence has 6-His amino acid at the C-terminus for purification. The target protein size from this construct is ˜132 kDa. The plasmid has two selection markers: kanamycin and puromycin.

FIG. 4: Expression of various constructs of recombinant dengue vaccine in CHO (Chinese Hamster Ovary) confirmed by Western Blot. DeN I (DeNFL), DeN II (DeNLC) and DeN III (DeNHC) are shown in the blot at ˜190 kDa, ˜58 kDa and ˜132 kDa, respectively.

FIG. 5A is Table 1, which lists the details of the vaccine constructs.

FIG. 5B is a continuation of Table 1 of FIG. 5A.

### DESCRIPTION OF THE SEQUENCE LISTING

SEQ ID NO: 1 is the amino acid sequence of the mutated light chain domain of DrTeNT(detoxified tetanus neurotoxin).

SEQ ID NO: 2 is the amino acid sequence of heavy chain domain of DrTeNT.

SEQ ID NO: 3 is the amino acid sequence of DrTeNT.

SEQ ID NO: 4 is the nucleotide sequence of DeNFL.

SEQ ID NO: 5 is the amino acid sequence of DeNFL.

SEQ ID NO: 6 is the nucleotide sequence of DeNLC.

SEQ ID NO: 7 is the amino acid sequence of DeNLC.

SEQ. ID NO: 8 is the nucleotide sequence of DeNHC.

SEQ. ID NO: 9 is the amino acid sequence of DeNHC.

SEQ ID NO: 10 is Epitope 1:AAYIVIGVGDSALGPGPGEGTDAPCKIPFSSQDEK

SEQ ID NO: 11 is Epitope II: GPGPGRHVLGRLITVNPVTEK.

SEQ ID NO: 12 is Epitope III: AAYRGMSYAMCTNTFVVLKKEVS.

SEQ ID NO: 13 is Epitope IV: AAYDSYIVIGVGNSALTL.

In one or more embodiments, the claimed inventions comprise a sequence having: at least 85%, at least 90%, or at least 95% sequence identity with the claimed SEQ ID NO.

### DETAILED DESCRIPTION

### Glossary of Terms

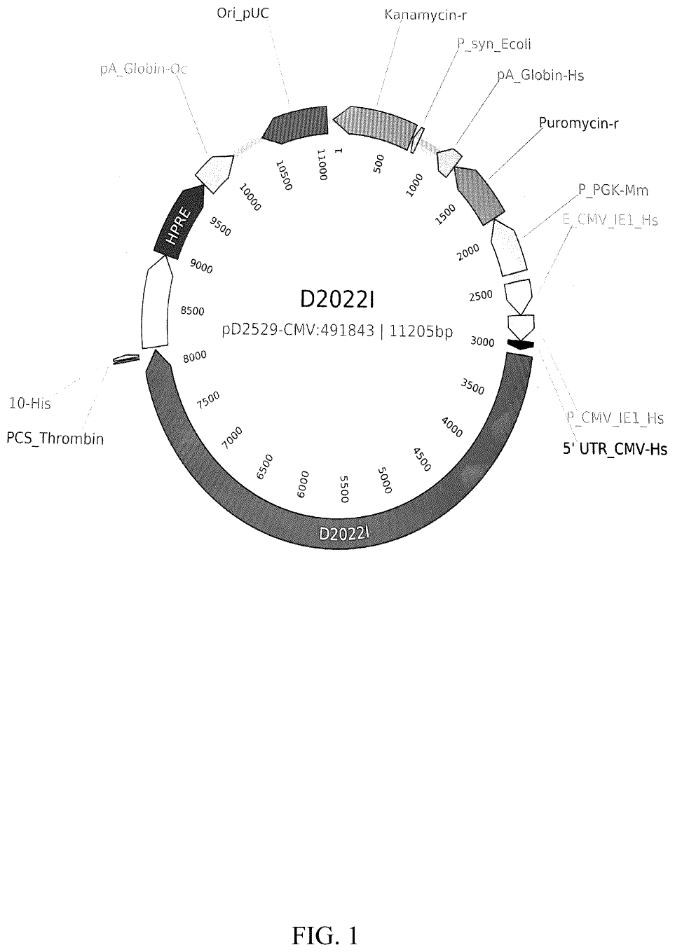

As used herein, “DeNFL” refers to the (plasmid) construct of FIG. 1, wherein the abbreviation stands for Dengue Vaccine Full Length (e.g., SEQ ID NOS: 4 and 5):

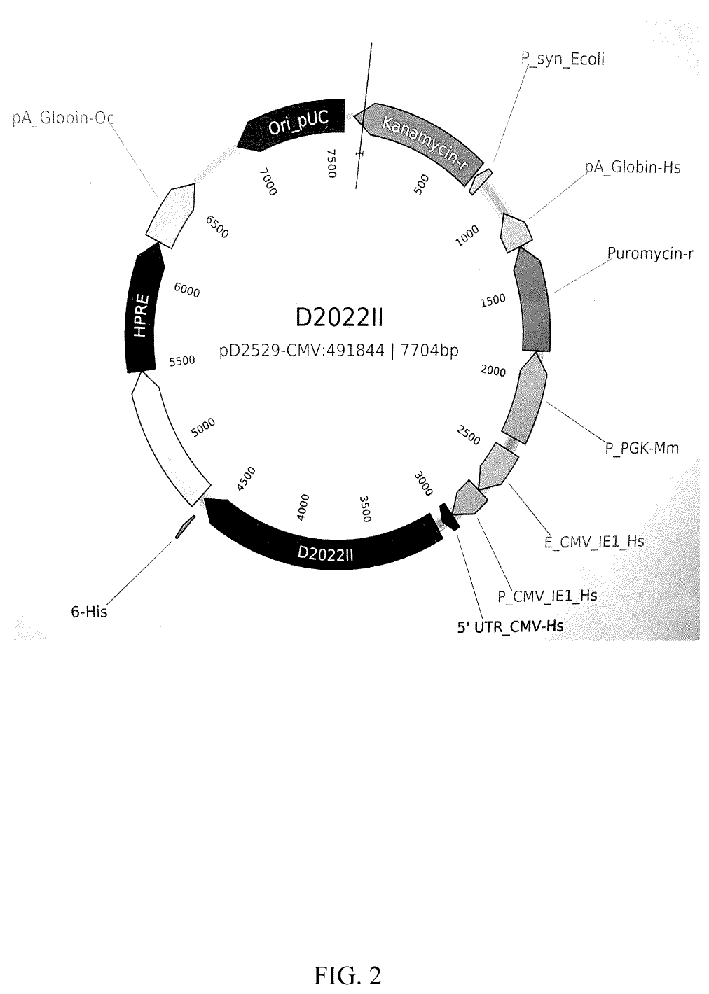

As used herein, “DeNLC” refers to the construct of FIG. 2, wherein the abbreviation stands for Dengue Vaccine Light Chain (e.g., SEQ ID NOS: 6 and 7):

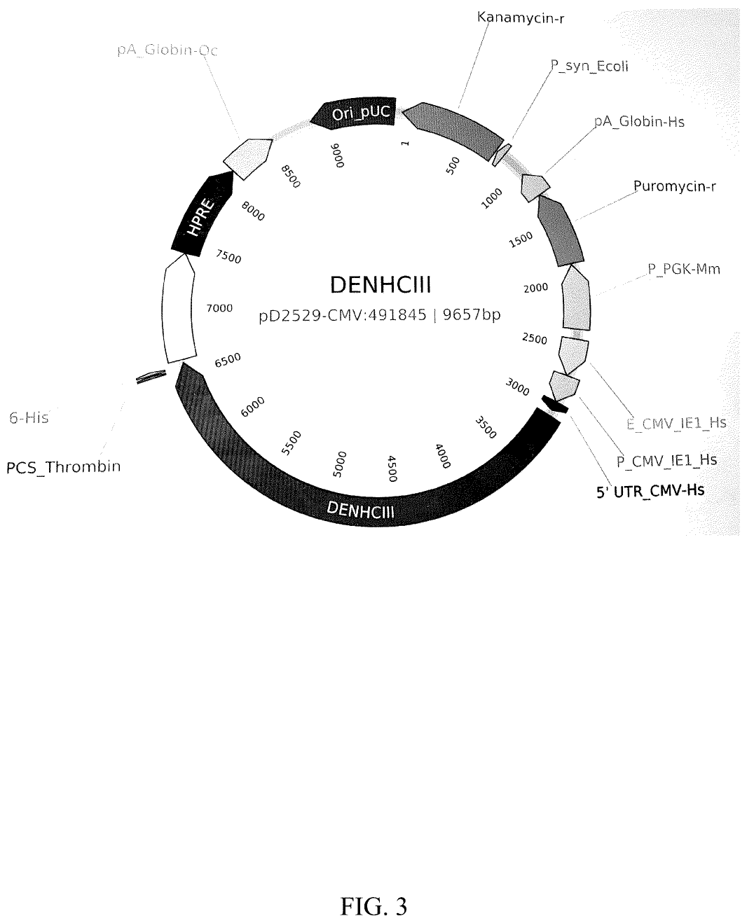

As used herein, “DeNHC” refers to the construct of FIG. 3, wherein the abbreviation stands for Dengue Vaccine Heavy Chain (e.g., SEQ ID NOS: 8 and 9):

As used herein, “DENV1, DENV2, DENV3” refers to dengue virus serotypes 1, 2, or 3.

Accordingly, the first aspect of the presently claimed invention is directed to a composition for induction of neutralizing antibodies and/or T-cells in a subject against two or more dengue virus serotypes comprising a protein obtained from: a vector comprising a nucleic acid sequence with at least 85%, 90%, or 95% sequence identity to SEQ ID NO: 4; and/or a vector comprising a nucleic acid sequence with at least 85%, 90%, or 95% sequence identity to SEQ ID NO: 6; and/or a vector with at least 85%, 90%, or 95% sequence identity to with a nucleic acid sequence comprising SEQ ID NO: 8.

Dengue infections are caused by four closely related viruses named DEN-1, DEN-2, DEN-3, and DEN-4. The distinctions of different serotypes are based on the different interactions with the antibodies in human blood serum. Although the genomes of all the serotypes are approximately 65% similar, there are

genetic variations in a single serotype. Overall amino acid residues among serotypes are ≥30%. Despite these variations, infection with each of the dengue serotypes results in the same disease and range of clinical symptoms. The dengue virus is a type of RNA virus. It is a single strand positive sense of RNA which can be directly translated as a single, long polypeptide, which is then processed into ten proteins of two types: a) three structural proteins: the capsid (C), envelope (E), and membrane (M) proteins; and b) seven non-structural proteins: NS1, NS2A, NS2B, NS3, NS4A, NS4B, and NS5. Dengue virus is spherically shaped with an approximate diameter of 50 nm. The core of the virus is the nucleocapsid surrounded by an envelope protein and a membrane protein that spans through the lipid bilayer. Dengue virus enters the host cell through endocytosis and utilizes the host cell's machinery to replicate its viral genome into proteins. After maturation, dengue viruses infect other host cells.

One of the approaches for an effective vaccine against dengue could be a multi-epitope vaccine. The present invention comprises designing a vaccine that will comprise strong epitopes of all four serotypes of dengue. These epitopes could be a combination of B-cell and T-cell, so that a balance between long term and protective immunity could be established. These epitopes are embedded in an already known vaccine backbone of the present inventor (DrTeNT-detoxified tetanus neurotoxin, as disclosed in US Published Patent Application US20190076518 published Mar. 14, 2019), which is established for long term protective antibody response with activated immune memory system. This is the basis of the technology according to the presently claimed invention.

As used herein, the term “effective immune response” comprises prevention of infection, and/or reduction in symptoms upon infection, by two or more serotypes of dengue virus in a human or non-human subject, especially mammalian. The immune response comprises neutralizing antibodies, or a T-cell response (killer T cells, and/or helper T cells), or a combination of these.

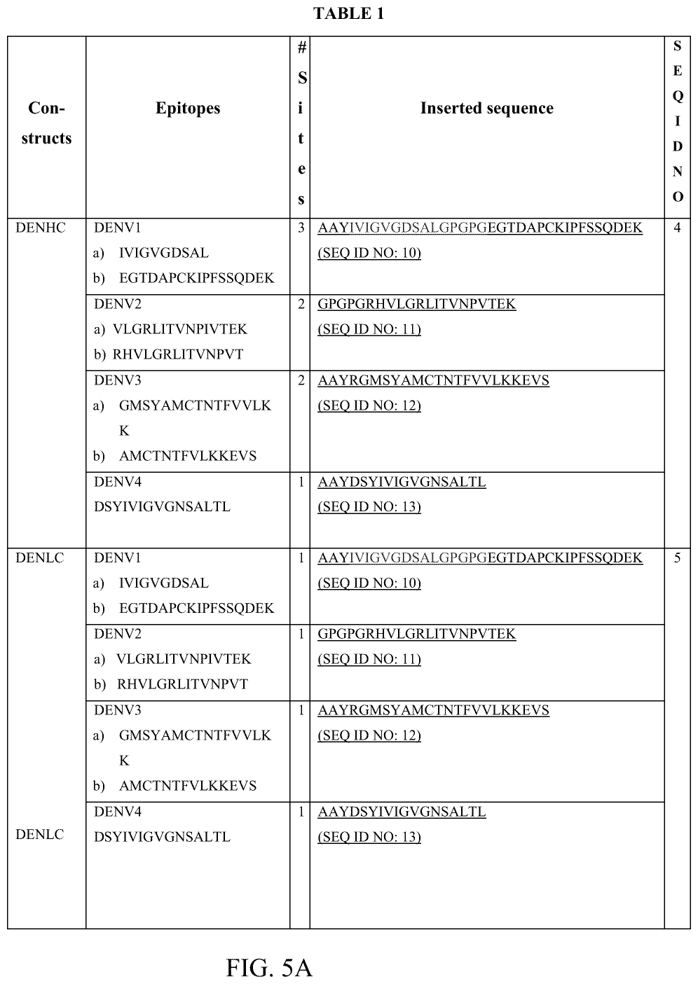

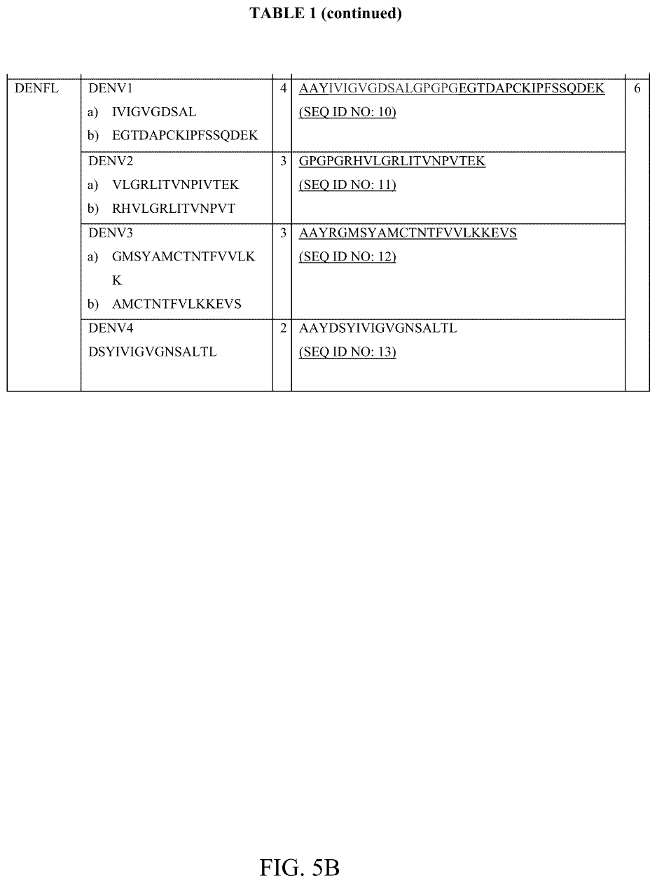

The B-cell and the T-cell epitopes were embedded (14-20 amino acid long) in a recombinant backbone platform of tetanus vaccine (DrTeNT: Detoxified recombinant Tetanus Neurotoxin). The epitopes were selected from a viral library for Dengue virus. For each serotype both B-cell and T-cell epitopes were selected. All the epitopes were selected from domain-III of envelop protein of all the serotypes of dengue virus. The two epitopes each of DENV1, DENV2 and DENV3, and one epitope of DENV4 are embedded into DrTeNT amino acid sequence and converted to corresponding nucleotide sequence (see FIGS. 5A, 5B: Table 1). The three different constructs were prepared using an optimized nucleotide sequence by inserting into the pD2529-CMV-491845 vector using appropriate restriction enzymes. The pD2529-CMV-491845 vector was equipped with proper enhancer/promoter and higher gene expression elements. Four epitopes were selected for creating three different sequences.

a. Epitope 1:
(SEQ ID NO: 10)
AAYIVIGVGDSALGPGPGEGTDAPCKIPFSSQDEK.
b. Epitope II:
(SEQ ID NO: 11)
GPGPGRHVLGRLITVNPVTEK.
c. Epitope III:
(SEQ ID NO: 12)
AAYRGMSYAMCTNTFVVLKKEVS.
d. Epitope IV:
(SEQ ID NO: 13)
AAYDSYIVIGVGNSALTL.

Construct I: The amino acid sequence of the LCT (Light chain of DrTeNT; SEQ ID NO: 1) was used as a backbone. LCT backbone was translated into nucleotide sequence. In the nucleotide backbone, one each of all the above epitopes were inserted at an appropriately optimized novel site to create DeNLC (D2022II) construct (SEQ ID NO: 6) (nucleotide sequence) or SEQ ID NO: 7 (amino acid sequence). SEQ ID NO: 6 was optimized for the CHO (Chinese Hamster Ovary) cells with kanamycin and puromycin as selection marker.

Construct II: The amino acid sequence of the HCT (SEQ ID NO: 2) was used as a backbone. LCT backbone was translated into nucleotide sequence. In the nucleotide backbone, epitopes of all the serotypes were inserted at an appropriately optimized novel site to create DeNHC (DeNHCIII) construct (SEQ ID NO: 8 (nucleotide sequence) or SEQ ID NO: 9 (amino acid sequence)). SEQ ID NO: 8 has four epitopes of DENV1, three each of DENV2 and DENV3, and two of DENV4. After embedding the SEQ ID NO: 8 was optimized for the CHO cells with kanamycin and puromycin as selection marker.

Construct III: The amino acid sequence of the DrTeNT (SEQ ID NO: 3) was used as a backbone. DrTeNT backbone was translated into nucleotide sequence. In the nucleotide backbone, epitopes of all the serotypes were inserted at an appropriately optimized novel site to create DeNFL (D2022I) construct (SEQ ID NO: 8 (nucleotide sequence) or SEQ ID NO: 9 (amino acid sequence)). SEQ ID NO: 8 has four epitopes of DENV1, three each of DENV2 and DENV3, and two of DENV4. After embedding the SEQ ID NO: 8 was optimized for the CHO cells with kanamycin and puromycin as selection marker.

All the above constructs were transfected in a mammalian suspension cell system. Briefly, the cells were grown to a viability of >95%. The cell count number was between 0.8-1.0×10⁷ cells/ml. The day before transfection, the cells were sub-cultured into a new flask to 3×10⁶ cells/ml and allowed to grow overnight. On the day of transfection, 0.8-1.0 μg/ml of DNA was diluted into media and mixed with lipofectamine. Lipid-DNA complex was mixed slowly into the cells and allowed to grow for 24 hr. The next day, enhancer and feed were added to the media and the temperature of the incubation was brought down to 32° C. and allowed to grow. Shaking speed was between 120-125 rpm. The cell count was taken every other day and on the 5^{th} day additional enhancer and feed were added. The cells were harvested on the day when the number of cells and/or viability was 3×10⁶ cells/ml and ≤70%, respectively.

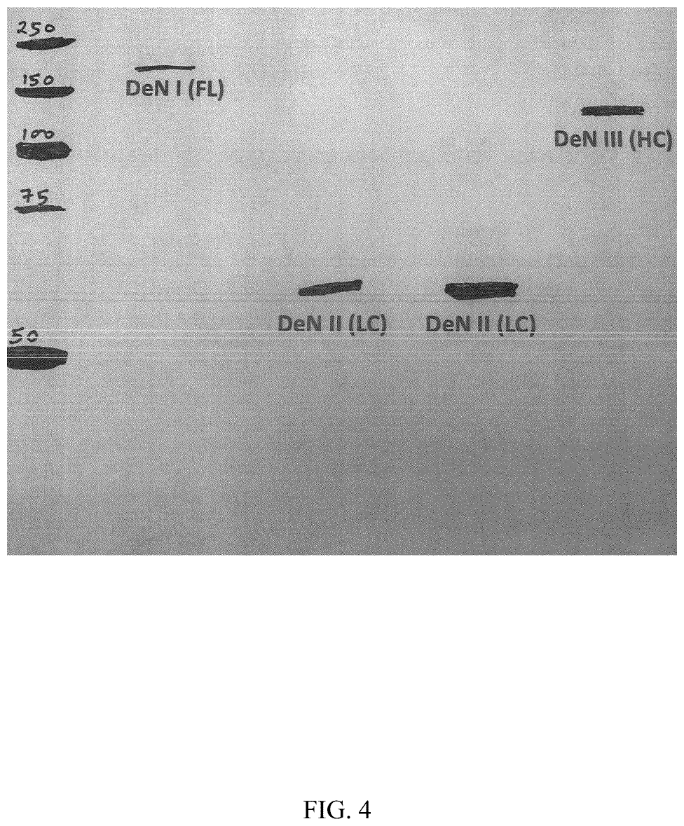

Expression of proteins was determined using Western Blot. Briefly, the cells were resuspended in 1×PBS and lysed by using M-per and sonication. The lysate was loaded in 12% SDS-PAGE gel and the proteins were separated and transferred into PVDF (Poly VinyliDene Fluoride) membrane using Bio Rad turbo blot. The membrane was blotted using anti-DrTeNT antibody produced in rabbit (1:1500) as a primary antibody (1.5 hr incubation) and HRP-conjugated anti-IgG (1:2500) as a secondary antibody (1.5 hr incubation). In between every incubation, the membrane was washed three times with PBST (0.05% tween-20 in 1×PBS containing 3% non-fat dry milk). Antibody solutions were also prepared in PBST. Finally, the membrane was developed using TMB (3,3′-5,5′ tetra methylbenzidine) solution (FIG. 4).

In an embodiment, the present invention is the composition of a protein-based vaccine against dengue that will induce neutralizing antibodies in a subject against two or more serotypes of dengue virus.

In another embodiment, the vaccine comprises of three different backbone sequences taken from Tetanus neurotoxin; mutated recombinant light chain of tetanus neurotoxin (˜50 kDa; SEQ ID NO: 1), recombinant heavy chain of tetanus neurotoxin (˜100 kDa; SEQ ID NO: 2), and detoxified mutated recombinant full length tetanus neurotoxin (˜150 kDa; SEQ ID NO: 3).

In another embodiment, the present invention is the selection of various linear epitopes (B- and T-cell epitopes) from all of the four serotypes. As mentioned above, epitopes are selected from envelope protein domain III.

In another embodiment, the present invention comprises the selection of the epitope insertion site in the backbone. This is selected (for all three backbone sequences) by using SVMTrip™ (Yao et al., 2012). Epitopes are selected and inserted as per the list in Table 1 (FIGS. 11A, 11B).

In another embodiment, the present invention comprises a recombinant gene comprising a nucleic acid sequence that encodes a polypeptide comprising SEQ ID NO: 4 along with an appropriate promoter/s, origin of replication and selection marker/s in a bacterial or mammalian or yeast or insect or viral vector (FIG. 1).

In another embodiment, the present invention comprises a recombinant gene comprising a nucleic acid sequence that encodes a polypeptide comprising SEQ ID NO: 6 along with an appropriate promoter/s, origin of replication and selection marker/s in a bacterial or mammalian or yeast or insect or viral vector (FIG. 2).

In another embodiment, the recombinant gene comprising a nucleic acid sequence that encodes a polypeptide comprising SEQ ID.—8 along with an appropriate promoter/s, origin of replication and selection marker/s in a bacterial or mammalian or yeast or insect or viral vector (FIG. 3).

In another embodiment, the present invention comprises of inserting the vector carrying recombinant genes corresponding to SEQ ID. 4, 6 and 8.

In another embodiment, the present invention comprises of expressing the recombinant vaccines corresponding to SEQ ID NOS: 4,5: 6, 7; and 8, 9.

In another embodiment, the present invention comprises of molecules with at least 95% sequence identity to SEQ ID NOS: 4,5: 6, 7; and 8, 9. In another embodiment, the present invention comprises of epitopes with at least 85% sequence identity of inserted sequences of Table 1.

In another embodiment, the present invention comprises of four inserted amino acid sequences:

(SEQ ID NO: 10)
AAYIVIGVGDSALGPGPGEGTDAPCKIPFSSQDEK;
(SEQ ID NO: 11)
GPGPGRHVLGRLITVNPVTEK;
(SEQ ID NO: 12)
AAYRGMSYAMCTNTFVVLKKEVS;
and
(SEQ ID NO: 13)
AAYDSYIVIGVGNSALTL.

In another embodiment, the present invention comprises expressing these different constructs in an appropriate expression system, which includes: bacterial, mammalian, yeast, insects or any other expression system (FIG. 4).

In another embodiment, the present invention comprises the purification of different constructs using affinity, ion exchange and gel filtration chromatography.

In another embodiment, the present invention comprises the formulation of protein vaccines for subcutaneous, oral, sublingual or nasal administration.

In another embodiment, the present invention comprises of treating and/or preventing dengue either by purified protein or inserting the vector or formulated vaccines.

In another embodiment, the present invention comprises one or more immunomodulators along with vaccines or vaccine carrying vector or epitopes carrying molecule.

In another embodiment, the present invention comprises one or more epitopes that should be recognized by antigen presenting cells and processes it for desire immune response.

In another embodiment, the present invention comprises formulating the purified protein/s by neurotoxin associated proteins (NAPs) or Hemagglutinin proteins or ORFs proteins from Botulinum neurotoxin complex to either protect from harsh environment of digestive/nasal tract, or to increase the bioavailability, or both.

In another embodiment, the present invention comprises one or more adjuvants such as alum, polylactic acid, polyglycolic acid, vitamins, colloidal particles, non-ionic surfactants. etc.

In another embodiment, the present invention comprises of administering one or more than one protein or vectors or genes of SEQ ID NOS: 4, 5; 6, 7; 8 and 9.

In another embodiment, the present invention comprises a vaccine vehicle that will be able to activate the memory cells and prime the system for eliciting the desired immune response.

In another embodiment, the present invention further comprises one or more compositions comprising, or containing, or consisting of, or consisting essentially of: a vaccine that can be used to effectively immunize human and/or non-human mammals against dengue virus.

### CONCLUSION

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

The transitional term “comprising”, which is synonymous with “including,” “containing,” or “characterized by,” is inclusive or open-ended and does not exclude additional, unrecited elements or method steps. The transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

Or, the technology illustratively described herein suitably may be practiced in the absence of any element(s) not specifically disclosed herein. Thus, for example, in each instance herein any of the terms “comprising,” “consisting essentially of,” and “consisting of” may be replaced with either of the other two terms.

The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 5% of the underlying parameter (i.e., plus or minus 1-5%),

As used herein, the term “substantially” refers to approximately the same shape as stated, or synonymous with “about 100%” or the like indicating certainty.

While several embodiments of the disclosure have been described, it is not intended that the disclosure be limited thereto, as it is intended that the disclosure be as broad in scope as the art will allow and that the specification be read likewise. Therefore, the above description should not be construed as limiting, but merely as exemplifications of embodiments.

Trademarks: the product names used in this document are for identification purposes only; and are the property of their respective owners.

### LIST OF REFERENCES CITED

Sustaining the drive to overcome the global impact of neglected tropical diseases: second WHO report on neglected tropical diseases. WHO (2013) WHO/HTM/NTD/2013.1
L. M. Ling, A. Wilder-Smith, and Y. S. Leo. Fulminant hepatitis in dengue haemorrhagic fever. Journal of Clinical Virology, vol. 38, no. 3, pp. 265-268, 2007.
S. Gulati and A. Maheshwari. Atypical manifestations of dengue. Tropical Medicine and International Health, vol. 12, no. 9, pp. 1087-1095, 2007.
R. Kumar, S. Tripathi, J. J. Tambe, V. Arora, A. Srivastava, and V. L. Nag. Dengue encephalopathy in children in Northern India: clinical features and comparison with non-dengue. Journal of the Neurological Sciences, vol. 269, no. 1-2, pp. 41-48, 2008.
M. Wasay, R. Channa, M. Jumani, G. Shabbir, M. Azeemuddin, and A. Zafar, “Encephalitis and myelitis associated with dengue viral infection. Clinical and neuroimaging features,” Clinical Neurology and Neurosurgery, vol. 110, no. 6, pp. 635-640, 2008.
T. Solomon, N. M. Dung, D. W. Vaughn et al., “Neurological manifestations of dengue infection,” The Lancet, vol. 355, no. 9209, pp. 1053-1059, 2000.
S. B. Park, S. Y. Ryu, K. B. Jin et al., “Acute colitis associated with dengue fever in a renal transplant recipient,” Transplantation Proceedings, vol. 40, no. 7, pp. 2431-2432, 2008.
R. Edelman, S. S. Wasserman, S. A. Bodison et al., “Phase I trial of 16 formulations of a tetravalent live-attenuated dengue vaccine,” The American Journal of Tropical Medicine and Hygiene, vol. 69, no. 6, pp. 48-60, 2003.
Zhang Y, Corver J. Chipman P R, Zhang W. Pletnev S V, Sedlak D. Baker T S. Strauss J H, Kuhn R J, Rossmann M G. Structures of immature flavivirus particles. EMBO) J. 2003 Jun. 2; 22(11):2604-13.
Modis Y, Ogata S, Clements D. Harrison S C. A ligand-binding pocket in the dengue virus envelope glycoprotein. Proc Natl Acad Sci USA. 2003 Jun. 10; 100(12):6986-91.
Kuhn R J, Zhang W, Rossmann M G, Pletnev S V, Corver J, Lenches E, Jones C T, Mukhopadhyay S. Chipman P R, Strauss E G, Baker T S, Strauss J H. Structure of dengue virus: implications for flavivirus organization, maturation, and fusion. Cell. 2002 Mar. 8; 108(5):717-25.
Chen R F, Yang K D, Wang L. Liu J W, Chiu C C. Cheng J T. Different clinical and laboratory manifestations between dengue haemorrhagic fever and dengue fever with bleeding tendency. Trans R Soc Trop Med Hyg. 2007 November; 101(11):1106-13.
Leng C H. Liu S J, Tsai J P, Li Y S, Chen M Y, Liu H H, Lien S P, Yueh A, Hsiao K N, Lai L W, Liu F C, Chong P, Chen H W. A novel dengue vaccine candidate that induces cross-neutralizing antibodies and memory immunity. Microbes Infect. 2009 February; 11(2):288-95
Henchal E A, Putnak J R. The dengue viruses. Clin Microbiol Rev. 1990 October; 3(4):376-96.
S. Simasathien, S. J. Thomas, V. Watanaveeradej et al., “Safety and immunogenicity of a tetravalent live-attenuated dengue vaccine in flavivirus naive children,” The American Journal of Tropical Medicine and Hygiene, vol. 78, no. 3, pp. 426-433, 2008.
W. Sun, D. Cunningham, S. S. Wasserman et al., “Phase 2 clinical trial of three formulations of tetravalent live-attenuated dengue vaccine in flavivirus-naïve adults,” Human Vaccines, vol. 5, no. 1, pp. 33-40, 2009.
Yao B, Zhang L, Liang S, Zhang C. SVMTriP: a method to predict antigenic epitopes using support vector machine to integrate tri-peptide similarity and propensity. PLoS One. 2012; 7(9):e45152. doi:10.1371/journal.pone.0045152.
Zhang R, Hryc C F, Cong Y, Liu X, Jakana J, Gorchakov R, Baker M L, Weaver S C, Chiu W. 4.4 Å cryo-EM structure of an enveloped alphavirus Venezuelan equine encephalitis virus. EMBO J. 2011 Aug. 9; 30(18):3854-63.
Crill W D, Roehrig J T. Monoclonal antibodies that bind to domain III of dengue virus E glycoprotein are the most efficient blockers of virus adsorption to Vero cells. J Virol. 2001 August; 75(16):7769-73.
Thullier, Philippe, Caroline Demangel, Hugues Bedouelle, Françoise Mégret, Alain Jouan, Vincent Deubel. Jean-Claude Mazié, and Pierre Lafaye. “Mapping of a dengue virus neutralizing epitope critical for the infectivity of all serotypes: insight into the neutralization mechanism.” Journal of General Virology 82, no. 8 (2001): 1885-1892.
Modis Y, Ogata S, Clements D. Harrison S C. Variable surface epitopes in the crystal structure of dengue virus type 3 envelope glycoprotein. J Virol. 2005 January; 79(2):1223-31.
Wahala W M, Kraus A A, Haymore L B, Accavitti-Loper M A, de Silva A M. Dengue virus neutralization by human immune sera: role of envelope protein domain III-reactive antibody. Virology. 2009 Sep. 15; 392(1):103-13.
Li X Q, Qiu L W, (Chen Y, Wen K, Cai J P, Chen J, Pan Y X, Li J, Hu D M, Huang Y F, Liu L D, Ding X X, Guo Y H, Che X Y. Dengue virus envelope domain III immunization elicits predominantly cross-reactive, poorly neutralizing antibodies localized to the AB loop: implications for dengue vaccine design. J Gen Virol. 2013 October; 94(Pt 10):

What is claimed is:

1. A pharmaceutical composition for eliciting an effective immune response in a patient against two or more dengue virus serotypes, the composition comprising one or more amino acid sequences encoded by one or more of:
a vector comprising a nucleic acid sequence with at least 90% sequence identity to SEQ ID NO: 4; and/or
a vector comprising a nucleic acid sequence with at least 90% sequence identity to SEQ ID NO: 6; and/or,
a vector comprising a nucleic acid sequence with at least 90% sequence identity to SEQ ID NO: 8; and,
wherein expression of the amino acid sequences encoded by the one or more vectors elicits a neutralizing antibody titer against dengue virus serotypes DENV-1, DENV-2, DENV-3 and/or DENV-4.

2. The pharmaceutical composition of claim 1, further comprising one or more epitopes or domains of dengue virus comprising an amino acid sequence with at least 90% sequence identity to SEQ ID NOS: 1, 2, or 3 to obtain the one or more amino acid sequences encoded with at least 90% sequence identity to SEQ ID NOS: 5, 7,9.

3. The pharmaceutical composition of claim 1, wherein the vector comprises a nucleic acid sequence having at least 95% sequence identity with SEQ ID NO: 4, 6, or 8.

4. The pharmaceutical composition of claim 1, wherein the nucleic acid sequence is expressed in one or more of: bacteria, yeast, insects, mammalian or any other appropriate expression system.

5. The pharmaceutical composition of claim 1, further comprising one or more proteins derived from Botulinum Neurotoxin (BoNT) produced by a Clostridium botulinum, the one or more proteins comprising: a neurotoxin associated proteins (NAPs); a hemagglutinin (Hn); an open reading frame (Orf); or a combination thereof.

6. The pharmaceutical composition of claim 1, further comprising an adjuvant comprising one or more of: alum, polylactic acid, polyglycolic acid, vitamins, colloidal particles, and non-ionic surfactants.

7. The pharmaceutical composition of claim 1, further comprising one or more of: nanoparticles, emulsions, lipids, liposomes, microspheres, and bioenhancers.

8. The pharmaceutical composition in claim 1, wherein the patient is a human or a non-human vertebrate.

9. The pharmaceutical composition of claim 1, wherein the composition is able to be administered as a protein, and/or DNA or plasmid molecule.

10. The composition of claim 9, wherein the plasmid molecule is administered alone or in combination with one or more adjuvants via a route of administration comprising: intraperitoneal (IP), subcutaneous (SubQ), intramuscular (IM), oral, buccal, sub lingual, nasal or topical route.

11. A method of inducing protection against two or more serotypes of dengue virus in a patient, comprising administering to a patient a first composition comprising one or more DNA vectors or proteins of claim 1.

12. The method of inducing protection of claim 11, wherein the first composition comprises one or more proteins encoded by the nucleotide sequence of SEQ ID NOS: 4, 6, or 8, and the one or more proteins that are purified out to form a stable second composition that is administered to a patient.

13. The method of inducing protection of claim 12, wherein a method of purifying proteins from the first composition to form the stable second composition comprises affinity chromatography, ion exchange and gel filtration chromatography.

14. The method of inducing protection of claim 12, wherein the stable second composition is administered to a patient, and elicits neutralizing B-cell and T-cell responses in the patient against at least two dengue virus serotypes.

15. The method of inducing protection of claim 11, wherein the route of administration comprises: intraperitoneal (IP), subcutaneous (SubQ), intramuscular (IM), oral, buccal, sub lingual, nasal or topical route.

16. A method of producing one or more proteins able to elicit an effective immune response against two or more dengue virus serotypes, comprising the steps of:
i. providing a vector, comprising a nucleotide sequence of:
Dengue Vaccine Full Length (DeNFL), or
Dengue Vaccine Light Chain (DeNLC), or
Dengue Vaccine Light Chain (DeNHC),
wherein each construct comprises one or more epitopes of SEQ ID NOS: 10-13inserted into a plasmid to obtain an expression vector comprising a nucleotide sequences with at least 85% sequence identity to SEQ ID NOS: 4, 6, and/or 8;
ii. obtaining a protein from the expression vector, the protein comprising an amino acid sequence at least 85% homologous to SEQ ID NOS: 5, 7, and/or 9.

17. The method of producing a protein of claim 16, wherein the vector is the construct of FIG. 1 (DeNFL), FIG. 2 (DeNLC), or FIG. 3 (DeNHC).

18. The method of producing a protein of claim 16, wherein the protein vaccine comprises the amino acid sequence with at least 85% sequence identity of SEQ ID NO: 5, 7 and/or 9.

19. The method of producing a protein of claim 16, wherein the epitope comprises the amino acid sequence with at least 95% sequence identity to one or more of: DENV1 (SEQ ID NO: 10); DENV2 (SEQ ID NO: 11); DENV3 (SEQ ID NO: 12); and DENV4 (SEQ ID NO: 13).

20. The method of producing a protein of claim 16, wherein the protein of SEQ ID NO: 5, 7 and 9 are expressed in vivo or in vitro.
